(19) United States
(12) Patent Application Publication
(10) Pub. No.: US 2003/0219476 A1
(43) Pub. Date: Nov. 27, 2003

(54) Liposomal formulation of mitoxantrone

(75) Inventors: Imran Ahmad, Wadsworth, IL (US); Aquilur Rahman, Potomac, MD (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: NeoPharm, Inc., Lake Forest, IL (US)

(21) Appl. No.: 10/413,061

(22) Filed: Apr. 14, 2003

Related U.S. Application Data
(63) Continuation of application No. PCT/US01/42757, filed on Oct. 16, 2001.
(60) Provisional application No. 60/241,069, filed on Oct. 16, 2000.

(51) Int. Cl. A61K 9/127; A61K 31/135

(52) U.S. Cl. 424/450; 514/649

(57) ABSTRACT

This invention pertains to liposomal formulations of mitoxantrone and methods for their manufacture and use. The compositions of the present invention include liposomal formulations of mitoxantrone in which the liposome contains any of a variety of neutral or charged liposome-forming materials in addition to a compound that is thought to bind mitoxantrone, such as cardiolipin. The liposomal compositions can be used advantageously in conjunction with secondary therapeutic agents other than mitoxantrone, including antineoplastic, antifungal, antibiotic among other active agents. Methods are provided in which a therapeutically effective amount of the formulation is administered to a mammal, such as a human.

### BACKGROUND OF THE INVENTION

This invention pertains to liposomal formulations of mitoxantrone and methods for their manufacture and use.

### DESCRIPTION OF THE BACKGROUND

Mitoxantrone, especially its hydrochloride salt form, is a therapeutic agent which is useful for the treatment of cancer and multiple schlerosis. The U.S. Food and Drug Administration (FDA) first approved mitoxantrone hydrochloride for sale in the United States in 1987 as an injectable formulation under the tradename Novantrone®. Novantrone® is provided as a sterile, nonpyrogenic, dark blue aqueous solution containing an amount of the hydrochloride salt form equivalent to 2 mg/ml mitoxantrone free base, with sodium chloride (0.80% w/v), sodium acetate (0.005% w/v), and acetic acid (0.046% w/v) as inactive ingredients.

Novantrone® in combination with corticosteroids is approved for use as initial chemotherapy for the treatment of patients with pain related to advanced hormone-refractory prostate cancer. The recommended dosage of Novantrone is 12 to 14 mg/m² given as a short intravenous infusion every 21 days.

Novantrone is also approved for use, in combination with other approved drug(s), in the initial therapy of acute nonlymphocytic leukemia (ANLL), including myelogenous, promyelocytic, monocytic, and erythroid acute leukemias. The recommended dosage is 12 mg/m² of Novantrone daily on days 1-3 given as an intravenous infusion along with 100 mg/m² of cytarabine for 7 days given as a continuous 24-hour infusion on days 1-7.

Novantrone® is also approved for use in reducing neurologic disability and/or the frequency of clinical relapses in patients with secondary (chronic) progressive, progressive relapsing, or worsening relapsing-remitting multiple sclerosis. Mitoxantrone hydrochloride is thought to be a DNA-reactive agent that is cytotoxic to both proliferating and non-proliferating human cells in culture.

The toxicity of mitoxantrone limits the dosage of drug that can be administered to patients. Moreover, the development of multidrug resistance in cells exposed to mitoxantrone can limit its effectiveness. Consequently, formulations of mitoxantrone are needed that sufficiently solubilize mitoxantrone while maximizing its efficacy for example, by minimizing toxicity and the development of multidrug resistance in treated cells.

The present invention provides such a composition and methods. These and other advantages of the present invention, as well as additional inventive features, will be apparent from the description of the invention provided herein.

### SUMMARY OF THE INVENTION

The present invention is for novel mitoxantrone compositions, their preparation methods, and their use in treating diseases such as cancer, particularly in mammals, especially humans. The method involves administering a therapeutically effective amount of the pharmaceutical composition of mitoxantrone in a pharmaceutically acceptable excipient to a mammal. The compositions of the present invention include liposomal formulations of mitoxantrone in which the liposome can contain any of a variety of neutral or charged liposome-forming materials and a compound such as cardiolipin that is thought to bind mitoxantrone. The liposome-forming material can be an amphiphilic molecule such as a phospholipid like phosphatidyl choline, dipalmitoyl phosphatidyl choline, phosphatidyl serine, cholesterol, and the like that form liposomes in polar solvents. The cardiolipin in the liposomes can be derived from natural sources or synthetic. Depending on the composition of the liposomes, the liposomes can carry net negative or positive charges or can be neutral. Preferred liposomes also contain tocopherol. Although a wide range of concentrations of mitoxantrone can be used in this formulation, the most useful concentrations range from 0.5 to 2 mg/ml. The molar ratio of the mitoxantrone to lipid component can also vary widely but the most useful range is from about 1:10 to about 1:20. The liposomes can be passed through filters of various sizes to control their size, as desired. The liposomal compositions can be used advantageously in conjunction with secondary therapeutic agents other than mitoxantrone, including antineoplastic, antifungal, antibiotic among other active agents. The liposomes of the present invention can be multilamellar vesicles, unilamellar vesicles, or their mixtures, as desired. Methods are provided in which a therapeutically effective amount of the present liposomes in a pharmaceutically acceptable excipient are administered to a mammal, such as a human.

In one particularly preferred method of manufacturing the dosage form, a quantity of mitoxantrone in a pharmaceutically acceptable excipient (such as Novantrone®, is added to a vessel containing a quantity of preformed lyophilized liposomes that contain a mitoxantrone-binding component, and the mitoxantrone is allowed to bind to the liposomes to provide the pharmaceutical dosage form.

### DETAILED DESCRIPTION OF THE PREFERRED EMBODIMENTS

The present invention provides a composition and methods for its manufacture and delivery to a mammalian host. The composition and method are characterized by avoidance of solubility problems of mitoxantrone, high mitoxantrone and liposome stability, ability to administer mitoxantrone as a bolus or short infusion in a high concentration, reduced mitoxantrone toxicity, particularly reducing mitoxantrone accumulation in cardiac muscle, increased therapeutic efficacy of mitoxantrone, and modulation of multi-drug resistance in cancer cells. The use of cardiolipin in the formulation improves mitoxantrone entrapment to a surprising extent.

The inventive composition is a liposomal formulation of mitoxantrone which contains cardiolipin. Generally, the liposomal formulation can be prepared by known techniques. For example, in one preferred technique mitoxantrone is dissolved in a hydrophobic solvent with cardiolipin and the cardiolipin allowed to form complexes with mitoxantrone. The cardiolipin/mitoxantrone-containing mixture can be evaporated to form a film in order to facilitate complex formation. Thereafter, solutions containing any desired additional lipophilic ingredients can be added to the film and the mitoxantrone/cardiolipin complexes dissolved or thoroughly dispersed in the solution. The solution can then be evaporated to form a second lipid film. A polar solvent, such as an aqueous solvent, can then be added to the lipid film and the resulting mixture vigorously homogenized to produce the present inventive liposomes.

Alternatively, all of the lipophilic ingredients can be dissolved in a suitable solvent that can then be evaporated to form a lipophilic film. A polar solvent such as an aqueous solvent can then be added to the lipid film and the resulting mixture vigorously homogenized to produce the present inventive liposomes.

Where the mitoxantrone is dissolved in the lipid film, as described above, the dosage form can be conveniently packaged in a single vial to which a suitable aqueous solution can be added to form the liposomes. Altematively, a two vial system can be prepared in which the lipophilic ingredients or preformed liposomes are contained in one vial and aqueous ingredients containing mitoxantrone are provided in a second vial. The aqueous mitoxantrone-containing ingredients can be transferred to the vial containing the lipid film or preformed liposomes and the liposomal formulation of mitoxantrone formed by vigorous mixing, vortexing and/or sonicating.

Desirably, the liposomes, once formed, are filtered through suitable filters to control their size. Suitable filters include those that can be used to obtain the desired size range of liposomes from a filtrate. For example, the liposomes can be formed and thereafter filtered through a 5 micron filter to obtain liposomes having a diameter of about 5 microns or less. Alternatively, 1 μm, 500 nm, 200 nm, 100 nm, or other filters can be used to obtain liposomes having corresponding sozes.

To prepare the mitoxantrone formulation mitoxantrone is dissolved in a suitable solvent. Suitable solvents are those in which mitoxantrone is soluble and which can be evaporated without leaving pharmaceutically unacceptable amounts of pharmaceutically unacceptable residue. For example, non-polar, slightly polar, or polar solvents can be used, such as ethanol, methanol, chloroform, acetone, or saline, and the like.

Any suitable cardiolipin can be used in the present invention. For example, cardiolipin can be purified from natural sources or can be chemically synthesized, such as tetramyristylcardiolipin. Cardiolipin can be dissolved in a suitable solvent, which include solvents in which cardiolipin is soluble and which can be evaporated without leaving pharmaceutically unacceptable amounts of pharmaceutically unacceptable residues. The cardiolipin solution can be mixed with the mitoxantrone. Alternatively, cardiolipin can be dissolved directly with mitoxantrone. It has been found that by incorporating cardiolipin in liposomes, the liposomes capacity for mitoxantrone is increased to a surprising extent. Thus, suitable cardiolipin derivatives can also be used in the present liposome formulation so long as the resulting liposome formulation is sufficiently stable for therapeutic use and has a suitable capacity for mitoxantrone.

Any suitable liposome-forming material can be used in the present liposomal formulation. Suitable liposome-forming materials include synthetic, semi-synthetic (modified natural) or naturally occurring compounds having a hydrophilic portion and a hydrophobic portion. Such compounds are amphiphilic molecules and can have net positive, negative, or neutral charges. The hydrophobic portion of liposome forming compounds can include one or more nonpolar, aliphatic chains, for example, palmitoyl groups. Examples of suitable liposome-forming compounds include phospholipids, sterols, fatty acids, and the like. Preferred liposome-forming compounds include cardiolipin, phosphatidyl choline, cholesterol, dipalmitoyl phosphatidyl choline, phosphatidyl serine, and α-tocopherol.

The liposome-forming material can be dissolved in a suitable solvent, which can be a low polarity solvent such as chloroform, or a non-polar solvent, such as n-hexane, in which it is soluble. Suitable solvents only include solvents in which the liposome-forming material is soluble and which can be evaporated without leaving pharmaceutically unacceptable amounts of pharmaceutically unacceptable residues. Other components can be mixed in with this solution, including mitoxantrone, to form a solution in which all ingredients are soluble and the solvent can then be evaporated to produce a homogeneous lipid film. Solvent evaporation can be by any suitable means that preserves the stability of mitoxantrone and other lipophilic ingredients.

Suitable liposomes can be neutral, negatively, or positively charged, the charge being a function of the charge of the liposome components and pH of the liposome solution. For example, at neutral pH, positively charged liposomes can be formed from a mixture of phosphatidyl choline, cholesterol, and stearyl amine. Negatively charged liposomes can be formed, for example, from phosphatidyl choline, cholesterol, and phosphatidyl serine. In a preferred embodiment, the liposomal mitoxantrone formulation contains tetramyristoyl cardiolipin, cholesterol, and egg phosphatidylcholine.

The preferred liposomal mitoxantrone formulation contains suitable relative molar amounts of mitoxantrone to lipid. Suitable relative molar amounts of mitoxantrone to lipid range of about 1:1-50, more preferably, about 1:2-40, more preferably about 1:5-30, still more preferably about 1:10-20, and most preferably about 1:15.

The liposomal formulation also contains suitable relative molar amounts of cardiolipin, phosphatidylcholine, and cholesterol. Suitable relative molar amounts include about 0.1-25:1-99:0.1-50 of cardiolipin:phosphatidylcholine:cholesterol. More preferably, relative molar amounts range from 0.2-10:2-50:1-25, still more preferably 0.5-5:4-25:2-15, and still more preferably the amounts range from 0.75-2:5-15:4-10, the most preferred ratio being 1:10:6.8. Preferred liposomal formulations also contain suitable amounts of antioxidants such as α-tocopherol or other suitable antioxidants. Suitable amounts range from about 0.001 or more to about 5 wt. % or less.

Liposomes can be formed by adding a polar solution preferably an aqueous solution, such as a saline solution, to the lipid film and dispersing the film with vigorous mixing. Preferably, the polar solution contains mitoxantrone. The solution can be pure water or it can contain salts, buffers, or other soluble active agents. Any method of mixing can be used provided that the chosen method induces sufficient shearing forces between the lipid film and polar solvent to strongly homogenize the mixture and form liposomes. For example, mixing can be by vortexing, magnetic stirring, and/or sonicating. Multilamellar liposomes can be formed simply by vortexing the solution. Where unilamellar liposomes are desired a sonication and/or filtration step can be included in the process.

In the preferred method of manufacturing the liposomal mitoxantrone formulation, a vial of lyophilized liposomes is prepared and Novantrone® is added to form the liposomal formulation of the mitoxantrone. The lyophilized liposomes are manufactured by dissolving the lipid ingredients and D-α-tocopheryl acid in warm butyl alcohol as described in more detail in Example 7. Warm water with trehalose dihydrate is mixed into this solution until the solution is clear. The solution is sterile filtered through a 0.22 μm filter into sterile vials and lyophilized. Desirably, the lyophilized product is an off-white cake or powder having a moisture content of about 12% or less and that can easily be reconstituted into a uniform solution of liposomes having a pH of from about 3 to about 6.

The final dosage form is prepared by adding 7.5 ml of a mitoxantrone solution (15 mg) such as from a Novantrone® vial and 7.5 ml of normal saline (0.9% NaCl) to a vial of the lyophilized lipids. The liposome mixture hydrates at room temperature for 30 minutes and is vortexed vigorously for 2 minutes at room temperature. The mixture is allowed to hydrate while being sonicated at maximum intensity for 10 minutes in a bath-type sonicator. This final dosage form may be dispensed in either a syringe or standard infusion set over 45 minutes for use within 8 hours after reconstitution. Using this method about 70 wt. % or more of the added mitoxantrone can be entrapped in the liposomal formulation. More preferably, about 80 wt. % or more of the mitoxantrone is entrapped. More preferable, about 85 wt. % or more of the mitoxantrone is entrapped in liposomes. Still more preferably, about 90 wt. % or more or even about 95 wt. % or more of mitoxantrone is entrapped in the liposomes.

The efficiency of mitoxantrone entrapment can be determined by dialysis of an aliquot of the liposomal preparation overnight in an aqueous solution and thereafter dissolving the liposomes in methanol and analyzing the sample by standard methods using high pressure reverse phase liquid chromatography (HPLC). Alternatively, liposomes can be collected after centrifugation at 50,000×g for 1 hour prior to dissolving them in methanol for HPLC analysis. Generally the encapsulation efficiency of mitoxantrone in liposomes will be more than 80% of the initial input dose.

More generally, any suitable method of forming liposomes can be used so long as it results in liposomal mitoxantrone. Thus, solvent evaporation methods that do not involve formation of a dry lipid film can be used. For example, liposomes can be prepared by forming an emulsion in an aqueous and organic phase and evaporating the organic solvent. The present invention is intended to encompass liposomal formulations of mitoxantrone however made.

The invention includes pharmaceutical preparations which in addition to non-toxic, inert pharmaceutically suitable excipients contain the liposomal mitoxantrone formulation and processes for production of these preparations. By pharmaceutically suitable excipients there are to be understood solid, semi-solid or liquid diluents, fillers and formulation auxiliaries of all kinds. The invention also includes pharmaceutical preparations in dosage units. This means that the preparations are in the form of individual parts, for example vials, syringes, capsules, pills, suppositories, or ampoules, of which the content of liposomal entrapped mitoxantrone corresponds to a fraction or a multiple of an individual dose. The dosage units can contain, for example, 1, 2, 3 or 4 individual doses or ½, ⅓ or ¼ of an individual dose. An individual dose preferably contains the amount of mitoxantrone which is given in one administration and which usually corresponds to a whole, a half or a third or a quarter of a daily dose.

Tablets, dragees, capsules, pills, granules, suppositories, solutions, suspensions and emulsions, pastes, ointments, gels, creams, lotions, powders and sprays can be suitable pharmaceutical preparations. Suppositories can contain, in addition to the liposomal mitoxantrone, suitable water-soluble or water-insoluble excipients. Suitable excipients are those in which the inventive liposomal mitoxantrone is sufficiently stable to allow for therapeutic use, for example polyethylene glycols, certain fats, and esters or mixtures of these substances. Ointments, pastes, creams and gels can also contain suitable excipients in which the liposomal mitoxantrone is stable.

The mitoxantrone formulation should preferably be present in the abovementioned pharmaceutical preparations in a concentration of about 0.1 to 50, preferably of about 0.5 to 25, wt. % of the total dry formulation.

The abovementioned pharmaceutical preparations are manufactured in the usual manner according to methods as are known, for example, by mixing the liposomal mitoxantrone with the excipient or excipients.

The active compound and pharmaceutical preparations containing the active compound are used in human and veterinary medicine for the prevention, amelioration and/or cure of diseases, in particular those diseases caused by cellular proliferation, such as cancer, in any mammal, such as a cow, horse, pig, dog or cat. For example, dog lymphoma can be treated effectively with the present mitoxantrone formulation. However, the present formulation is particularly preferred for use in the treatment of human patients, particularly for cancer and other diseases caused by cellular proliferation. The inventive compositions have particular use in treating human multiple schlerosis, lymphoma, and prostate, liver, ovarian, breast, lung and colon cancers.

The active compound or its pharmaceutical preparations can be administered locally, orally, parenterally, intraperitoneally and/or rectally, preferably parenterally, however intravenous administration is preferred.

In a human of about 70 kg body weight, for example, from about 0.5-100 mg/m² mitoxantrone is administered. Preferably, from about 5.0 or more to 50 mg/m² of mitoxantrone or more preferably from about 10 or more to about 45 mg/m² is administered. Still more preferably about 20 or more to about 40 mg/m² and still more preferably about 25 or more to about 40 mg/m² of mitoxantrone can be administered. However, it can be necessary to deviate from the dosages mentioned and, in particular, to do so as a function of the nature and body weight of the subject to be treated, the nature and the severity of the illness, the nature of the preparation and if the administration of the medicine, and the time or interval over which the administration takes place. Thus, it can suffice in some cases to manage with less than the abovementioned amount of active compound while in other cases the abovementioned amount of active compound can be exceeded. Suitable amounts are therapeutically effective amounts that do not have excessive toxicity, as determined in empirical and case-by-case studies.

One advantage of the present composition is that it provides a method of modulating multidrug resistance in cancer cells that are subjected to mitoxantrone treatment. In particular, the present liposomal formulations reduce the tendency of cancer cells subjected to chemotherapy with mitoxantrone to develop resistance thereto, and reduces the tendency of treated cells of developing resistance to other therapeutic agents, such as camptothecin, taxol, or doxorubicin, for example. Thus, other agents can be advantageously employed with the present treatment either in the form of a combination active with mitoxantrone or by separate administration.

The examples demonstrate that mitoxantrone administration produces pharmacological efficacy against mammalian tumors that is not diminished by inclusion in a liposomal formulation. Further, animals could tolerate higher doses of mitoxantrone when it is administered as a liposomal formulation and they have better outcomes as measured by median survival times or reduced tumor volumes than animals given conventional mitoxantrone. Higher plasma concentrations in mice and dogs and a longer elimination half-life of compound in mice is demonstrated. Peak plasma concentrations were approximately 50-fold higher in the mouse and 9-fold higher in the dog at comparable dosages. Mouse tissue concentrations of conventional mitoxantrone were lower in heart, lung and kidneys and higher in liver and spleen after administration of liposomal mitoxantrone as compared to conventional mitoxantrone. Toxicity did not occur until higher doses of liposomal mitoxantrone were administered as compared to conventional mitoxantrone alone, however, toxicity profiles appear similar. No toxicity occurred in the liposomal formulation that has not been observed previously with mitoxantrone alone. In animals, higher doses of liposomal mitoxantrone are better tolerated and more effective than conventional mitoxantrone in its current conventional (non-liposomal) formulation.

Having described the present invention, reference will now be made to certain examples which are provided solely for purposes of illustration and which are not intended to be limiting.

### EXAMPLE 1

This example shows one formulation of liposomal mitoxantrone. Mitoxantrone (3 μmoles) is dissolved with cardiolipin in (3 μmoles) in chloroform. Phosphatidyl choline (14 μmoles) dissolved in hexane and 10 μmoles cholesterol in chloroform is added to the mitoxantrone mixture with stirring. The solvents are evaporated under vacuum at about 30° C. or below to form a thin dry film of lipid and drug. Liposomes are formed by adding 2.5 ml of saline solution and aggressively mixing the components, as by vortexing. The flasks can then be vortexed to provide multilamellar liposomes or sonicated to provide small unilamellar liposomes.

### EXAMPLE 2

This example demonstrates the preparation of another formulation of liposomal mitoxantrone. A solution of about 6 μM mitoxantrone, 6 μM cardiolipin, 28 μM phosphatidyl choline and 20 μM cholesterol is prepared in a suitable solvent which is then evaporated. The dried lipid/drug film is dispersed in a 7% aqueous trehalose-saline solution. The mixture is vortexed and sonicated. The liposomes can then be dialyzed, as desired. Mitoxantrone encapsulation is 80% or more as assayed by HPLC.

### EXAMPLE 3

This example demonstrates the preparation of another formulation of liposomal mitoxantrone. Mitoxantrone can be entrapped in liposomes by using 3 μM of the drug, 15 μM of dipalmitoyl phosphatidyl choline, 1 μM cardiolipin, and 9 μM cholesterol in a volume of 2.5 ml. The drug and lipid mixture can be evaporated under vacuum and resuspended in an equal volume of saline solution. Liposomes are prepared as described in Example 1. The mitoxantrone encapsulation efficiency is higher than 80% in this system.

### EXAMPLE 4

This example demonstrates the preparation of another formulation of liposomal mitoxantrone. In this preparation of liposomes 2 μM mitoxantrone, 2 μM of phosphatidyl serine, 11 μM phosphatidyl choline, 2 μM cardiolipin, and 7 μM cholesterol are dissolved in a solution. Lipiosomes are prepared as in Example 1. Greater than 80% mitoxantrone encapsulation efficiency can be expected.

### EXAMPLE 5

This example demonstrates another formulation of liposomal mitoxantrone. Mitoxantrone (3 μmoles) can be dissolved in chloroform containing 3 μmoles cardiolipin and the mixture allowed to form complexes. To facilitate complex formation the chloroform solvent is removed by evaporation. Phosphatidyl choline (14 μmoles) dissolved in hexane and 10 μmoles cholesterol in chloroform can be added to the dry film. The mixture is stirred gently and the solvents evaporated under vacuum at below 30° C. to form a thin dry film of lipid and drug. Liposomes are then formed by adding 2.5 ml of saline solution and aggressively mixing the components by vortexing. The flasks can then be vortexed to provide multilamellar liposomes and optionally sonicated in a sonicator to provide small unilamellar liposomes.

### EXAMPLE 6

This example demonstrates another formulation of liposomal mitoxantrone. Generally this method involves the steps of obtaining a mitoxantrone solution, adding the mitoxantrone solution to preformed liposomes and allowing the mixture to equilibrate such that liposomal mitoxantrone forms. Each vial of Novantrone® contains mitoxantrone hydrochloride equivalent to 2 mg/ml mitoxantrone free base, sodium chloride (0.8% w/v), sodium acetate (0.005% w/v) and acetic acid (0.046% w/v). The Novantrone® solution has a pH of 3.0 to 4.5 and contains 0.14 mEq of sodium per ml.

Preformed liposomes are prepared by adding about 2 g of D-α-tocopherol acid succinate to about 10 kg of t-butyl alcohol which is warmed to about 35-40° C. The solution is mixed for about 5 minutes until the tocopherol is dissolved. About 60 g of tetramyristoyl cardiolipin is added to the solution and the solution is mixed for about 5 minutes. About 100 g of cholesterol is added to the solution and the solution is mixed for about 5 more minutes then about 300 g of egg phosphatidyl choline is added and mixed for another 5 min. A second aqueous solution containing 2,000 g of water at about 35° C.- 40° C. and about 120 g of trehalose dihydrate is mixed into the lipid solution until the mixture is clear. The mixture is sterile filtered through a 0.22 micron pore size Durapore® Millipak 200 filter and about 11 g is filled into sterile vials and lyophilized. Liposomes prepared in this manner are in the form of an off-white cake or powder and are easily reconstituted. The moisture content of the lyophilized liposomes is about 12% or less. The lyophilized product is stored at 4° C. prior to use.

To prepare liposomal mitoxantrone 7.5 ml mitoxantrone solution (15 mg) from a Novantrone® vial is added to a vial of lyophilized lipids along with 7.5 ml of normal saline (0.9% NaCl). The vial is swirled gently, allowed to hydrate at room temperature for 30 minutes, vortexed vigorously for 2 min, and sonicated for 10 min in a bath-type sonicator at maximum intensity. Doses can then be withdrawn from the vial for use. The product may be dispensed in either a syringe or standard infusion set over 45 min. Desirably, the liposomal mitoxantrone is maintained at room temperature until use, and is used within 8 h of reconstitution.

### EXAMPLE 7

This example demonstrates another formulation of liposomal mitoxantrone. A lyophilized lipid composition containing cardiolipin:phosphatidylcholine:cholesterol in a 1:10:6.8 molar ratio was prepared. Twenty-nine trials were conducted varying the mitoxantrone to lipid molar ratios, hydration and sonication times. Formulations were dialyzed against normal saline overnight and the amount of mitoxantrone retained in each formulation was determined.

The study demonstrated that a mitoxantrone to lipid molar ratio of 1:15 (2 mg of 1,1,2,2 tetramyristoyl cardiolipin, 12 mg phosphatidylcholine, and about 4 mg cholesterol per mg of mitoxantrone) with hydration for 2 h and sonication for 10 min produced retained 94±3% liposomal mitoxantrone for a 1 mg/ml mitoxantrone solution, 95±6% liposomal mitoxantrone for a 2 mg/ml mitoxantrone solution, and 97% mitoxantrone for a 1.5 mg/ml mitoxantrone solution. Reduction of the hydration time to 30 min did not appear to significantly affect the amount of mitoxantrone retained in the formulation at the 1 mg/ml mitoxantrone concentration. Unless noted otherwise, in the following examples a 1 mg/ml mitoxantrone formulation was prepared with a 1:15 mitoxantrone to lipid molar ratio, a hydration time of 2 h, and a sonication time of 10 min.

### EXAMPLE 8

The following example demonstrates that mitoxantrone in the liposomal formulation described above has a lower toxicity as compared to identical concentrations of nonliposomal (conventional) mitoxantrone and that at least 15 mg/kg of mitoxantrone administered in a liposomal formulation is not toxic to mice. Eighty male CD2F1 mice weighing 20-22 g were acclimated for 1 week and randomly separated into 8 groups of ten animals each with 5 animals per cage. On day 0 all groups of animals were injected i.v. in the tail vein with the drug or vehicle control. The volumes administered were varied based on individual animal weights. Mouse weights were recorded for each mouse on alternate days following injection and observation for clinical illness were recorded at least daily. The injections were as shown in Table 1.

TABLE 1

| Group | Drug formulation | Dose |
|---|---|---|
| 1 | Conventional Mitoxantrone | 15 mg/kg |
| 2 | Conventional Mitoxantrone | 10 mg/kg |
| 3 | Conventional Mitoxantrone | 5 mg/kg |
| 4 | Liposomal mitoxantrone | 15 mg/kg |
| 5 | Liposomal mitoxantrone | 10 mg/kg |
| 6 | Liposomal mitoxantrone | 5 mg/kg |
| 7 | Blank liposomes | 15 mg/kg |
| 8 | Normal saline solution | |

In the first 5 days no adverse clinical side effects were manifested for any of the animals. During days 6-10 all group 1 animals became moribund. One such animal died on day 9 and the remaining group 1 animals were sacrificed on day 10. Four animals each from groups 4, 7, and 8 were sacrificed intentionally and blood hematology and clinical chemistry were studied. The major organs were also fixed in buffered 10% formalin and studied. No clinical signs of toxicity were apparent in any group other than group 1. Following the study all remaining animals were sacrificed and blood hematology and clinical chemistry were studied and the major organs were fixed in buffered 10% formalin and studied.

A comparison of weights seen in the various groups showed clinically moderate or unapparent changes for all groups except group 1, (15 mg/kg dose) of conventional mitoxantrone. The group 1 animals progressively lost weight up to about 35% by day 9/10. Group 2 animals initially showed significant weight loss of 20% by day 10 but gradually recovered throughout the remainder of the study. The remaining groups all gained weight steadily throughout the study.

In this and the following examples blood was analyzed for bilirubin, blood urine nitrogen (BUN), creatinine, alkaline phosphatase, aspartate aminotransferase (AST), alanine aminotransferase (ALT), hemoglobin, hematocrit, white blood cell count, red blood cell count, mean corpuscular volume (MCV), mean corpuscular hemoglobin (MCH), mean corpuscular hemoglobin concentration (MCHC), platelets, neutrophils, band neutrophils, lymphocytes, monocytes, eosinophils, basophils. Clinically significant elevations in ALT were noted in most of the group 1 mice and one of the group 7 mice at day 10. Similar AST elevations were also observed. Two group 1 mice also exhibited modest elevations in BUN but not creatinine, suggesting a prerenal effect, possibly caused by dehydration or hemoconcentration. No other drug related effects were observed in these studies.

Histopathology demonstrated compound effects on hematopoietic and lymphoid tissues of the spleen and bone marrow in mice treated with conventional mitoxantrone and liposomal mitoxantrone. Full recovery was seen on Day 67 in the liposomal mitoxantrone treated animals at all dose levels suggesting liposomal mitoxantrone was less cytotoxic.

In conclusion, no morbidity or mortality was seen in the study with any of the controls or the liposomal formulation of up to 15 mg/kg mitoxantrone whereas 100% morbidity was observed in the 15 mg/kg dose of conventional mitoxantrone HCl.

### EXAMPLE 9

The following example demonstrates that mitoxantrone in the liposomal formulation described in Example 7 has a lower toxicity as compared to identical concentrations of conventional mitoxantrone HCl and that up to 35 mg/kg of mitoxantrone can be administered to mice in a liposomal formulation without apparent toxicity. Twenty male CD2F1 mice weighing 20-22 g were acclimated for 1 week and randomly separated into 4 groups of five animals each with 5 animals per cage. On day 0 all groups of animals were injected i.v. in the tail vein with the drug or vehicle control. The volumes administered were varied based on individual animal weights. Mouse weights were recorded for each mouse on alternate days following injection and observation for clinical illness were recorded at least daily. The injections were as shown in Table 2.

TABLE 2

| Group | Drug formulation | Dose |
|---|---|---|
| 1 | Liposomal mitoxantrone | 35 mg/kg |
| 2 | Liposomal mitoxantrone | 25 mg/kg |
| 3 | Conventional Mitoxantrone | 25 mg/kg |
| 4 | Blank liposomes | 35 mg/kg |

In the first 5 days no adverse clinical side effects were manifested for any of the animals. During days 6-7 all group 3 animals became moribund. One such animal died on day 6 and the remaining group 3 animals were sacrificed on day 7. No clinical signs of toxicity were apparent in any other group. Following the study all remaining animals were sacrificed and blood hematology and clinical chemistry were studied as in Example 8. The major organs were fixed in buffered 10% formalin and studied in all deceased animals.

A comparison of weights seen in the various groups showed clinically moderate or unapparent changes for all groups except group 3 which received the 25 mg/kg dose of conventional mitoxantrone. The group 3 animals progressively lost weight up to about 30% by day 7. Group 1 animals initially showed significant weight loss of 20% by day 10 but gradually recovered throughout the remainder of the study. The remaining groups all gained weight steadily throughout the study.

In conclusion, no morbidity or mortality was seen in the study with the vehicle control or the liposomal formulation of mitoxantrone whereas 100% morbidity was observed in the 25 mg/kg dose of conventional mitoxantrone.

### EXAMPLE 10

The following example demonstrates that mitoxantrone in the liposomal formulation described in Example 7 has a lower toxicity as compared to identical concentrations of conventional mitoxantrone HCl and that at least 35 mg/kg of mitoxantrone administered in a liposomal formulation is not toxic to mice. Seventy male CD2F1 mice weighing 20-22 g were acclimated for 1 week and randomly separated into 7 groups of ten animals each with 5 animals per cage. On day 0 all groups of animals were injected i.v. in the tail vein with the drug or vehicle control. The volumes administered were varied based on individual animal weights. Mouse weights were recorded for each mouse on alternate days following injection and observation for clinical illness were recorded at least daily. The injections were as shown in Table 3.

TABLE 3

| Group | Drug formulation | Dose |
|---|---|---|
| 1 | Conventional Mitoxantrone | 10 mg/kg |
| 2 | Conventional Mitoxantrone | 25 mg/kg |
| 3 | Liposomal mitoxantrone | 10 mg/kg |
| 4 | Liposomal mitoxantrone | 25 mg/kg |
| 5 | Liposomal mitoxantrone | 35 mg/kg |
| 6 | Blank liposomes | 35 mg/kg |
| 7 | Normal saline solution | |

In the first 2 days no adverse clinical side effects were manifested for any of the animals. During day 3 all group 2 animals became moribund and 3 were sacrificed. Three animals each from groups 1,3,4,5,6, and 7 were also sacrificed intentionally on day 3 and blood hematology and clinical chemistry were studied. Three additional animals from group 2 were moribund sacrifices at day 7 and 3 additional animals from groups 1, 3, 4, 5, 6, and 7 were also sacrificed. On day 10 the remaining group 2 animals had died. No other clinical signs of toxicity were observed through day 60. No clinical signs of toxicity were apparent in any group other than in group 2. Following the study all remaining animals were sacrificed and blood hematology and clinical chemistry testing, as described in Example 8 was undertaken. The major organs were fixed in buffered 10% formalin and studied in all deceased animals.

A comparison of weights seen in the various groups showed clinically moderate or unapparent changes for all groups except group 2 which received the 25 mg/kg dose of conventional mitoxantrone. The group 2 animals progressively lost weight up to about 27% by day 7. Group 1 and group 5 animals initially showed significant weight loss (13% and 8%, respectively) but gradually recovered throughout the remainder of the study. The remaining groups all gained weight steadily throughout the study.

On day 3 no consistent compound effects were noted in the clinical chemistry data, although one mouse dosed with 25 mg/kg conventional mitoxantrone (Group 2) and one mouse dosed with 35 mg/kg liposomal mitoxantrone (Group5) had modest increases in ALT activities. Cytotoxic effects on white blood cells were noted with most mice dosed with mitoxantrone but not in blank liposome-dosed mice.

On day 7 clinical chemistry data were inconclusive, although AST and ALT activities varied more widely and trended toward higher levels, consistent with some liver injury in some animals. Day 67 mice showed similar inconsistent increases, as did several of the mice treated with blank liposomes (group 6).

In conclusion, no morbidity or mortality was seen in the study with any of the controls or the liposomal formulation of mitoxantrone whereas 100% morbidity was observed in group 2 animals, which received 25 mg/kg of conventional mitoxantrone.

### EXAMPLE 11

The following example demonstrates that the administration of multiple doses of mitoxantrone, as prepared in Example 7, is better tolerated when a liposomal formulation is given as compared to identical concentrations of conventional mitoxantrone HCl and that at least 10 mg/kg of liposomal mitoxantrone administered repeatedly on 5 consecutive days is not toxic to mice. Forty male CD2F1 mice weighing 20-22 g were acclimated for 1 week and randomly separated into 8 groups of five animals each with 5 animals per cage. On day 0 all groups of animals were injected i.v. in the tail vein with the drug or vehicle control and once daily thereafter for a period of 5 days. The volumes administered were varied based on individual animal weights. Mouse weights were recorded for each mouse on alternate days following injection and observations of clinical illness were recorded at least daily. The injections were as shown in Table 4.

TABLE 4

| Group | Drug formulation | Dose |
|---|---|---|
| 1 | Conventional Mitoxantrone | 2.5 mg/kg |
| 2 | Conventional Mitoxantrone | 5.0 mg/kg |
| 3 | Conventional Mitoxantrone | 7.5 mg/kg |
| 4 | Liposomal mitoxantrone | 2.5 mg/kg |
| 5 | Liposomal mitoxantrone | 5.0 mg/kg |
| 6 | Liposomal mitoxantrone | 7.5 mg/kg |
| 7 | Blank liposomes | 7.5 mg/kg |
| 8 | Normal saline solution | |

No adverse clinical effects were observed in any of the mice in the first 5 days. On day 6 animals in groups 1, 2, 3, and 6 exhibited ruffled fur and hunching behavior. Two animals in groups 2 and 3 were moribund sacrifices. Two animals from each of the remaining groups were sacrificed for analysis. On day 7 a total of 3 animals from group 2 and 2 animals from group 3 became moribund sacrifices, one additional animal from group 3 was found deceased, and one animal from groups 6, 7, and 8 was sacrificed for hematological and clinical chemistry analysis. There was no indication of clinical toxicity observed in any of the remaining animals through day 60 at which time all animals were sacrificed. Blood samples were collected for hematological and clinical chemistry testing as described in Example 8, and major organs were fixed in buffered 10% formalin.

Comparison of the animal masses in various groups was interpreted as moderate, mild or unapparent except in groups 2 (5 mg/kg conventional mitoxantrone) and 3 (7.5 mg/kg conventional mitoxantrone). These animals showed progressive weight loss of about 25% by day 7. Group 1 (2.5 mg/kg conventional mitoxantrone) and group 6 (7.5 mg/kg liposomal mitoxantrone) animals initially lost about 28% of their mass but gradually recovered through the end of the study. Other treatment groups exhibited no change in mass during the study.

On day 7 the mice sacrificed from groups 6, 7, and 8 each exhibited a modest AST elevation. The mouse from group 8 also had increased alkaline phosphatase activity and the mice from groups 6 and 7 had reduced creatinine and alkaline phosphatase. Moribund-sacrificed mice from groups 2, 3, and 6 exhibited marked, clinically significant, compound related leukopenia with decreased neutrophils and lymphocyte counts, and a modest decrease in platelet count. Mice from groups 1, 4, 6, and 7 were analyzed at day 64 and exhibited moderate elevations in alkaline phosphatase and AST but where otherwise normal.

Histopathologic examination demonstrated hematopoietic and lymphoid depletion of spleen and bone marrow and villous and/or crypt atrophy in the intestines in all treatment groups. Liposomal mitoxantrone appeared to be less cytotoxic than conventional mitoxantrone for the spleen and much less cytotoxic for the intestinal epithelium. Some hepatocellular vacuolar degeneration was seen in the livers of several mice administered conventional mitoxantrone at 5 or 7.5 mg/kg. In contrast, minimal hepatocellular vacuolar degeneration was seen in one mouse given 5 mg/kg liposomal mitoxantrone and none of the mice given 7.5 mg/kg liposomal mitoxantrone. Both conventional mitoxantrone and liposomal mitoxantrone administration led to a depletion of osteoblasts and osteoclasts sufficient to impair longitudinal bone growth in many mice. Significant recovery of all effects was observed by day 64 in all the surviving mice given conventional mitoxantrone and in the mice given liposomal mitoxantrone at 2.5 mg/kg. Mice at 7.5 mg/kg liposomal mitoxantrone still had minimal histologic effects in the hematopoietic and lymphoid tissues on day 64.

Liposomal mitoxantrone appeared slightly less cytotoxic than conventional mitoxantrone for the spleen and much less cytotoxic for intestinal epithelium in spite of the tissue distribution findings that indicated greatly enhanced tissue concentrations of mitoxantrone. Some hepatocellular vacuolar degeneration was seen in the livers of several mice administered conventional mitoxantrone at 5 or 7.5 mg/kg. Minimal hepatocellular vacuolar degeneration was seen in only 1 mouse at 5 mg/kg liposomal mitoxantrone and none of the mice at 7.5 mg/kg.

In summary, no morbidity or mortality was seen in any group that received liposomal mitoxantrone or in the group that received 2.5 mg/kg of conventional mitoxantrone. In contrast, all of the animals in groups 2 (5 mg/kg conventional mitoxantrone) and 3 (7.5 mg/kg conventional mitoxantrone) died.

### EXAMPLE 12

The following example demonstrates that mitoxantrone in the liposomal formulation described in Example 7 has a lower toxicity as compared to identical concentrations of conventional mitoxantrone HCl and that at least 35 mg/kg of mitoxantrone administered in a liposomal formulation is not toxic to mice. Thirty male CD2F1 mice weighing 20-22 g were acclimated for 1 week and randomly separated into 6 groups of five animals each with 5 animals per cage. On day 0 all groups of animals were injected i.v. in the tail vein with the drug or vehicle control and once daily thereafter for a period of 5 days. The volumes administered were varied based on individual animal weights. Mouse weights were determined for each mouse on alternate days following injection and observation for clinical illness were recorded at least daily. The injections were as shown in Table 5.

TABLE 5

| Group | Drug formulation | Dose |
|---|---|---|
| 1 | Conventional Mitoxantrone | 2.5 mg/kg |
| 2 | Conventional Mitoxantrone | 5.0 mg/kg |
| 3 | Liposomal mitoxantrone | 5 mg/kg |
| 4 | Liposomal mitoxantrone | 7.5 mg/kg |
| 5 | Liposomal mitoxantrone | 10 mg/kg |
| 6 | Normal saline solution | |

No adverse clinical effects were observed in any of the mice in the first 5 days. Animals in groups 1, 2, and 5 exhibited ruffled fur and hunching behavior. On day 8, 3 animals from groups 2 and 5 were moribund sacrifices and 1 animal from group 5 was deceased. On day 8, 3 animals from group 6 were sacrificed for hematology and clinical chemistry. On day 10, 1 animal from group 2 was a moribund sacrifice and 1animal was deceased. One animal from group 5 was a moribund sacrifice on day 10. Three animals in group 1 were deceased by day 12. One animal in group 4 was deceased on day 18. There was no indication of clinical toxicity observed in any of the remaining animals through day 60 at which time all animals were sacrificed. Blood samples were collected for hematological and clinical chemistry testing as described in Example 8, and major organs were fixed in buffered 10% formalin.

The variation in animal weight in various groups was moderate, mild or unapparent except in groups 2 (5 mg/kg conventional mitoxantrone) and 5 (10 mg/kg liposomal mitoxantrone). These animals showed progressive weight loss of about 35% and 25%, respectively, by day 9. By day 13, group 1 (2.5 mg/kg conventional mitoxantrone), group 3 (5 mg/kg liposomal mitoxantrone), and group 4 (7.5 mg/kg liposomal mitoxantrone) animals initially lost about 30%, 7%, and 30% of their weight, respectively. Their weight gradually returned during the study. Other treatment groups exhibited no change in mass during the study.

No morbidity or mortality was seen in the vehicle control group or groups receiving up to 5 mg/kg (1 time on 5 consecutive days) of liposomal mitoxantrone. Morbidity of 60% was observed for animals treated with 2.5 mg/kg of conventional mitoxantrone. Morbidity of 20% was observed with animals treated with 7.5 mg/kg of liposomal mitoxantrone. Treatment with 10 mg/kg of liposomal mitoxantrone or 5 mg/kg of conventional mitoxantrone was lethal to 100% of the mice tested.

Moribund sacrificed animals from groups 2 (5 mg/kg conventional mitoxantrone) and 5 (10 mg/kg liposomal mitoxantrone) exhibited marked elevations in AST and ALT. In addition, bilirubin concentration in 3 of the 4 group 2 mice tested and 1 of the 4 group 5 mice was greater than in control mice. The moribund animals exhibited marked leukopenia with reduced neutrophils and lymphocytes. Modest variable decreases in platelet counts were also observed. Minimal increases in red blood cell count were also observed. Other parameters were not significantly affected. Mouse sacrificed at day 70 exhibited normal clinical chemistry but had low white blood cell counts. Lymphocytes and neutrophils were low in these mice. Other parameters were normal.

In the single-dose experiment in Example 8, a 15 mg/kg dose of conventional mitoxantrone but not liposomal mitoxantrone induced significant increases in ALT signifying acute liver injury, but a higher dose in Example 10 did not. Taking the multiple dose data into account, it is clear that conventional mitoxantrone has the potential to cause significant liver injury. Data from the terminal sacrifices suggest that significant recovery takes place, with little evidence of either toxicity or cytotoxicity.

Mice from the higher dose groups exhibited cytotoxic effects on white blood cells and platelets, with clear decreases in neutrophils and lymphocytes and modest decreases in platelets. In the lower dose groups the effects were much less marked. The data show that conventional mitoxantrone at 5 mg/kg/day and liposomal mitoxantrone at 10 mg/kg/day induced roughly equivalent acute liver injury, as evidenced by increased ALT, AST and bilirubin by day 8.

In summary, clinical pathology data from these studies show that liposomal mitoxantrone administered at 10 mg/kg/day is no more toxic than conventional mitoxantrone when administered at 5 mg/kg/day and significant recovery from toxic and cytotoxic effects was evident. The data show that liposomal mitoxantrone can safely be administered at amounts that are more than twice that considered safe for conventional mitoxantrone.

### EXAMPLE 13

The following example demonstrates that the liposomal mitoxantrone formulation described in Example 7 reaches higher plasma concentrations, has a longer half-life, and a slower clearance rate in mammalian blood than does mitoxantrone administered in a conventional formulation. Pharmacokinetic evaluation was performed in male CD2F1 mice, after single dose i.v. administration of conventional and liposomal mitoxantrone formulations at 5 mg/kg. Groups of four mice were sacrificed at 5 min., 15 min., 30 min., 1 h, 2 h, 4 h, 8 h, 24 h and 48 h after dosing and their blood and organs were collected and analyzed for mitoxantrone content.

Plasma and tissue samples were analyzed for mitoxantrone by reverse phase HPLC. Plasma samples (0.25 ml) were mixed with 0.5 ml of solution of 0.01 mg/ml hexanesulfonic acid, 0.5 mg/ml ascorbic acid, and 0.25 μg ametantrone as internal standard. After vortexing for 30 sec., 0.5 ml of 0.1 M borate buffer (pH 9.5) and 150 μl of 1 M sodium hydroxide was added and the solution vortexed again for 30 sec. The samples were extracted with 10 ml of dichloromethane on a horizontal shaker for 1 h and centrifuged for 15 min. at 3,000 rpm. The organic layer (9 ml) was separated and evaporated under nitrogen. Samples were reconstituted with 10 μl of mobile phase prior to HPLC analysis. Tissue samples were homogenized in 1 ml of solution containing 20% ascorbic acid in 0.1 M citrate buffer, pH 3.0, and extracted as described above. Mitoxantrone was separated by reverse-phase chromatography (Waters μBondapak® C-18) using a mobile phase of 33% acetonitrile, and 67% 0.16 M ammonium formate buffer, pH 2.7 delivered at a flow rate of 1 ml/min. Mitoxantrone was detected at 600 nm. The limit of sensitivity was 10 ng/ml.

Plasma Pharmacokinetic parameters were assessed by standard methods. The elimination rate constant (K) was calculated from the linear regression analysis of plasma concentration-time curve. The area under the curve (AUC_{0→∞}) was calculated using the linear trapezoidal method with extrapolation of the terminal phase to infinity (Cₗₐₛₜ/K), where Cₗₐₛₜ is the last measured concentration. Other parameters calculated were total body clearance (Cl) as Dose/AUC; volume of distribution (Vₐᵣₑₐ)=Cl/K; elimination half-life (t_{1/2})=0.693/Kₐᵣₑₐ.

In summary, following i.v. administration, liposomal mitoxantrone produced a significantly higher peak plasma concentration (50-fold) as compared to conventional mitoxantrone. The decrease in plasma concentration followed first-order kinetics with elimination half-life of 6.6 min. and 1 h for conventional and liposomal formulations, respectively. The AUC values and terminal elimination half-lives were Cₘₐₓ, AUC and t_{1/2} values after conventional mitoxantrone were 0.41 μg/ml, 0.14 μg•hr/ml and 0.11 hr, respectively, while these values were approximately 21 μg/ml, 28 μg•hr/ml, and 1 hr, for these same parameters after liposomal mitoxantrone administration. These increases could be explained by the decrease in both the clearance and the volume of distribution of the compound. The calculated total mitoxantrone clearance was substantially reduced with liposomal mitoxantrone (3 ml/min/kg) as compared to conventional mitoxantrone (600 ml/min/kg). The calculated volume of distribution was also markedly reduced for liposomal mitoxantrone (0.3 l/kg) versus conventional mitoxantrone (5.5 l/kg).

A similar pattern of clearance from the tissues was observed for the lungs and kidneys with conventional mitoxantrone tissue concentrations of approximately 20 and 40 μg/g in the lungs and kidneys, respectively and 13 and 16 μg/g in these same tissues after liposomal mitoxantrone administration. In the liver, mitoxantrone concentrations decreased gradually from approximately 19 to 2 μg/g after administration of conventional mitoxantrone while liver concentrations increased from approximately 25 to 37 μg/g at 4 hours after administration of liposomal mitoxantrone before declining very gradually to 30 μg/g at 48 hours. Lower peak concentrations of mitoxantrone were detected in the heart for the liposomal formulation (5.6 μg/g tissue) versus conventional mitoxantrone (11 μg/g tissue) 5 minutes after administration. The difference remained at least 2-fold for up to 48 hours after administration.

At all time points examined, heart, lung, and kidney concentrations of conventional mitoxantrone were higher for the conventional mitoxantrone-treated mice than for the liposomal mitoxantrone-treated mice. At all time points examined, spleen and liver concentrations were higher for the liposomal mitoxantrone-treated mice than for the conventional mitoxantrone-treated animals, demonstrating that the liposomal formulation shifts the distribution of the compound. Administration of conventional mitoxantrone lead to heart tissue concentrations of approximately 10 μg/g at 5 and 15 minutes after compound administration with the concentrations decreasing gradually to 5 to 6 μg/g at 24 and 48 hours. After liposomal mitoxantrone administration, heart mitoxantrone concentrations were about 6 μg/g at 5 minutes and the concentration decreased gradually to approximately 2μg/g at 24 to 48 hours. These data suggest the potential for decreased cardiac toxicity for liposomal mitoxantrone.

### EXAMPLE 14

This example demonstrates the efficacy of liposomal mitoxantrone, as prepared in Example 7, against human leukemia cells and demonstrates the increased efficacy of the liposomal formulation as compared to a conventional mitoxantrone formulation. Murine leukemia cells, L1210 leukemia cells were grown in the peritoneum of CD2F1 mice by three serial propagations (i.p.). Ascites developed within eight days of the last inoculation were used in the following experiments. Cytostatic activities of liposomal and conventional formulations of mitoxantrone against L1210 ascitic leukemia was determined. Animal group weights were determined three times a week and clinically morbid animals were humanely sacrificed. The surviving mice were observed daily for 60 days. Group survival times post i.v. treatment with single or multiple doses of the drug was indicative of the relative anti-tumor potencies of liposomal and conventional mitoxantrone.

Female CD2F1 mice were divided into eight groups of 10 animals and inoculated i.v. with 10,000 L1210 cells. Drug was administered twenty-four hours later. Conventional mitoxantrone was administered at doses of 5 and 10 mg/kg. Liposomal mitoxantrone was administered i.v. at 5, 10, 20 or 35 mg/kg doses as a single injection and the median survival time for each group was determined. Surviving animals were sacrificed on day 60 of the experiment. Blank liposomes equivalent to the 35, mg/kg dose and normal saline was also administered as controls.

The median survival time for untreated animals was 7 days. Animals treated with 5 mg/kg conventional mitoxantrone and liposomal mitoxantrone had median survivals of 12 and 13 days, respectively. The median survival time for animals given 10 mg/kg conventional mitoxantrone was 20 days, with 2/10 animals alive at day 60. The median survival time for animals treated with 10 mg/kg liposomal mitoxantrone was 27 days with 4/10 mice surviving to day 60. All animals treated with liposomal mitoxantrone at 20 mg/kg survived to day 60. At the highest dose of liposomal mitoxantrone tested, 35 mg/kg, 9/10 animals survived to Day 60, with one animal found dead on day 18, probably due to compound toxicity.

These single dose studies suggest liposomal mitoxantrone can be administered at higher doses than conventional mitoxantrone with an improved clinical outcome. In a murine model of leukemia, liposomal mitoxantrone improved the median survival of animals as compared to conventional mitoxantrone at comparable dosages and decreased compound-related mortality at both the same and higher dosages. These results suggest that it may be possible to administer higher dosages of mitoxantrone in the liposomal mitoxantrone formulation without enhancing the risk of toxicity. Mice tolerated liposomal mitoxantrone dosages of up to 20 mg/kg (60 mg/m²), and did not exhibit significant toxicity until liposomal mitoxantrone dosages of 35 mg/kg (105 mg/m²).

### EXAMPLE 15

This example demonstrates the efficacy of liposomal mitoxantrone, as prepared in Example 7, when administered in multiple doses. Forty female CD2F1 mice were separated into 4 groups of ten animals and inoculated with L1210 cells as described in Example 14. The mice were treated with conventional mitoxantrone at 2.5 mg/kg or liposomal mitoxantrone at 2.5 or 5 mg/kg every 24 hours for 4 days starting 24 hours after inoculation.

The median survival time for mice treated with conventional mitoxantrone and liposomal mitoxantrone at 2.5 mg/kg was 13 and 14 days, respectively. This survival time was similar to that described at the same concentration in the single dose study of Example 14. No animals survived to day 60 at this dose level in these treatment groups. Mice treated with liposomal mitoxantrone at 5 mg/kg had a median survival time of 37 days with 4/10 animals surviving to day 60. These data suggest a potential clinical benefit of liposomal mitoxantrone over conventional mitoxantrone when the drug is administered in multiple doses.

### EXAMPLE 16

This example demonstrates that in mice bearing xenografted human prostate cancer cells, survival was increased after single dose administration of liposomal mitoxantrone, as in Example 7, and mean tumor volume was reduced after multiple dose administration of liposomal mitoxantrone as compared to conventional mitoxantrone-treated animals. Male Balb/c, nu/nu, 6-8 week old mice were inoculated with 5×10⁶ of human hormone-refractory prostate tumor cells (PC-3). Tumor growth was monitored twice a week until the tumor volumes were in the range of 60-100 mm². Animals were then divided into groups and were treated by i.v. injection via the tail vein with conventional mitoxantrone at doses of 0.625, 1.25, 2.5, and 5 mg/kg once every other day for four days. Doses of mitoxantrone formulated in liposomes were 2.5, 5, 7.5, and 10 mg/kg. Control animals received either normal saline or blank liposomes. The median survival time was calculated and all surviving animals were sacrificed on Day 34.

Animals treated with conventional mitoxantrone at 0.625 and 1.25 mg/kg demonstrated 100% survival by day 34; however, no animals treated with 2.5 and 5 mg/kg survived. Survival rates for liposomal mitoxantrone were 100% for the 2.5 mg/kg dose, 91% for the 5 mg/kg dose, 43% for the 7.5 mg/ml dose, and 0% for 10 mg/kg dose.

The experiments were repeated for the treatments with conventional mitoxantrone at doses of 0.625 and 1.25 mg/kg and liposomal mitoxantrone at 2.5 and 5 mg/kg following the same dosing regimen. In these experiments tumor volumes were measured once or twice a week by measuring the three major axes.

Treatment with liposomal mitoxantrone at both dosages caused a significant reduction in tumor volume compared to control groups and treatment with conventional mitoxantrone. Significant delays in tumor growth were noted with PC-3 xenografts. Severe toxicity at the higher doses of conventional mitoxantrone limited its clinical usefulness. Liposomal mitoxantrone appears to be a safer more effective antitumor agent as compared to conventional mitoxantrone.

### EXAMPLE 17

This example demonstrates that the liposomal mitoxantrone formulation has a higher concentration in blood plasma, a slower clearance than conventional mitoxantrone following administration to dogs. Plasma samples from dogs (3/sex/group) administered conventional mitoxantrone i.v. at 0.13 or 0.26 mg/kg or liposomal mitoxantrone i.v. at 0.26, 0.58 or 0.87 mg/kg were analyzed for mitoxantrone levels by reverse phase HPLC using ametantrone as the internal standard. Time points analyzed were 0, 5 and 30 min and 1, 2, 4, 8 and 24 h after a single dose administration.

Plasma concentrations in animals receiving conventional mitoxantrone could not be measured at the 5 min time point for the low dose and 30 min for the high dose. One male that received 0.258 mg/kg was measurable at the 1 h time point. In contrast, most animals that received liposomal mitoxantrone had mitoxantrone plasma concentrations for up to 2 h for the low dose and 4 h for the mid and high doses.

Concentrations of mitoxantrone were much lower when conventional mitoxantrone was administered as compared to when liposomal mitoxantrone was administered as reflected in both Cₘₐₓ and AUC values (Table 6). Additionally, clearance was higher for conventional mitoxantrone as compared to liposomal mitoxantrone. Both Cₘₐₓ and AUC values increased with increasing liposomal mitoxantrone dosages while clearance, volume of distribution and elimination half-life were constant over the dosages. There were no differences in these parameters between the sexes. The results are summarized below in Table 6, which sets out the mean for each parameter. Other parameters shown in the table include the mitoxantrone half-life (t_{1/2}), the volume of distribution (V), and clearance rate (Cl).

TABLE 6

| | | Cₘₐₓ | AUC_{0→∞} | | Cl | |
|---|---|---|---|---|---|---|
| Mitoxantrone | Dose | (μg/ | t_{1/2} | (ml/ | V | |
| Formulation | (mg/kg) | ml) | hr/ml) | (hr) | min/kg) | (L/kg) |
| Conventional-M^{a} | 0.13 | 0.027 | NC^{b} | NC | NC | NC |
| Conventional-F | 0.13 | 0.016 | NC | NC | NC | NC |
| Conventional-M | 0.26 | 0.084 | 0.05^{c} | 0.36 | 89 | 2.8 |
| Conventional-F | 0.26 | 0.06 | NC | NC | NC | NC |
| Liposomal-M | 0.26 | 0.43 | 0.32 | NC | 17 | 1.2 |
| Liposomal-F | 0.26 | 0.77 | 0.42 | 0.25 | 15 | 0.9 |
| Liposomal-M | 0.58 | 1.5 | 0.84 | 0.3 | 13 | 0.6 |
| Liposomal-F | 0.58 | 1.9 | 1.7 | NC | 6.8 | 0.6 |
| Liposomal-M | 0.87 | 2.41 | 1.84 | NC | 9 | 0.8 |
| Liposomal-F | 0.87 | 2.33 | 1.77 | NC | 11 | 1 |

^{a}M = Male; F = Female
^{b}NC = Not Calculated
^{c}conventional mitoxantrone was detected only until the 30 minute sampling time

This example shows that in the dog, administration of liposomal mitoxantrone produced about a 9-fold increase in peak plasma mitoxantrone concentration as compared to identical doses of conventional mitoxantrone. The liposome formulation also exhibited increased AUC values as well as a decreased clearance rate. Both Cₘₐₓ and AUC values increased linearly with increasing dosage. The Cₘₐₓ values were approximately 0.5, 1.7 and 2.4 μg/ml at 0.26, 0.58 and 0.87 mg/kg (5, 12 and 17 mg/m²).

### EXAMPLE 18

This example demonstrates that dogs can tolerate higher doses of mitoxantrone when the drug is formulated in liposomes as compared to a conventional formulation of mitoxantrone. Conventional mitoxantrone was administered to beagle dogs (3/sex/group) at i.v. dosages of 0 (saline), 0.129 or 0.258 mg/kg (2.6 or 5 mg/m²) on Days 1, 23, 43 and 65. On these same days, beagle dogs (3/sex/group) received liposomal mitoxantrone at 0 (blank liposomes), 0.258, 0.580 or 0.869 mg/kg (5, 12 or 17 mg/m²). Evaluations for compound-related effects were based on clinical observations, body weight, food consumption, ophthalmologic and ECG examination, clinical pathology, plasma drug concentrations, organ weights and gross and microscopic postmortem examinations.

One male dog in the 0.869 mg/kg liposomal mitoxantrone group was sacrificed on Day 12 after one dose of liposomal mitoxantrone was administered due to lesions and swelling of the left limbs, hypoactivity, pallor, dehydration, and diarrhea.

One blank liposomal mitoxantrone treated female dog had alopecia while a second had excessive salivation through the first 29 days of the study. One female dog from the 0.869 liposomal mitoxantrone group was limping on its left side on Days 31, 32 and 36 and on Day 52, when it exhibited inflammation and swelling of the left hind foot along with a sore or ulcer on that foot.

None of the animals weight was affected during the study except for males in the 0.258 conventional mitoxantrone group who lost weight. There were no changes in food consumption in any groups.

There were no changes in ECG parameters at any examination time.

Animals administered 0.129 and 0.258 conventional mitoxantrone had leukopenia and thrombocytopenia 4 to 10 days following each dose cycle and the severity was dose related. White blood counts tended to rebound towards normal values during the latter half of the 3-week dosing cycle. The differential white blood cell data revealed a dose-related decline in neutrophil counts that was most severe on the 10^{th} day after each dose administration. Dose-related lymphopenia also occurred with each dosing cycle and appeared to worsen with each successive dose. Anemia did not occur in the conventional mitoxantrone animals but evidence of erythroid toxicity as evidenced by decreased reticulocyte counts was observed. Reticulocyte counts rebounded rapidly to normal or slightly higher than normal values on Days 10, 32, and 46.

Animals given liposomal mitoxantrone had changes in hematology parameters similar to those observed in the conventional mitoxantrone-treated animals with the exception that the animal sacrificed during the study (0.869 mg/kg liposomal mitoxantrone) had leukopenia, thrombocytopenia and anemia. A slight anemia was seen in the female dogs along with decreases in reticulocyte counts in both male and female dogs. Rebound of reticulocyte counts was not as fast in females as in male dogs.

No changes in coagulation or clinical chemistry parameters were observed for animals in any of the dosage groups.

At necropsy, 1 male in the liposomal mitoxantrone 0.869 mg/kg group had a fluid-filled pleural cavity and a thickened heart as well as gastrointestinal lesions. These findings appear to be compound-related. At this dosage one animal had discoloration of various lymph nodes. Three animals total in the liposomal mitoxantrone 0.580 and 0.869 groups had blue coloration at injection sites. No other findings were attributed to administration of compound.

In summary 1 of six dogs administered liposomes alone and 1 of 18 animals administered liposomal mitoxantrone had limb sores accompanied by limping, which is likely due to administration of the liposomes themselves. This study demonstrates that dogs can tolerate higher doses of mitoxantrone when the drug is formulated in liposomes.

### EXAMPLE 19

This example demonstrates a method for administering liposomal mitoxantrone to patients having cancer and a method for determining a safe and effective amount of a liposomal mitoxantrone formulation. Patients with histologically documented solid tumors are selected for treatment. In this study the maximum tolerated dose (MTD), dose limiting toxicity, and the blood pharmacokinetics of mitoxantrone following i.v. administration can be determined. Anti-tumor effects of liposomal mitoxantrone were also observed. Patients are treated with i.v. administration of liposomal mitoxantrone every three weeks until disease progression or occurrence of toxicity requiring early treatment termination was observed. The safety and tolerability of treatments are also determined. Pharmacokinetic parameters are assessed in the first course of therapy. Cardiac status is evaluated every second course. Disease status is assessed after every second course by appropriate means. Six dose levels are evaluated.

Commercial Novantrone® is used in this study. The liposomal formulation of mitoxantrone was prepared as described in Example 7. Liposomal mitoxantrone is administered i.v. over 45 min. at the doses shown below in Table 7.

TABLE 7

| | Liposomal Mitoxantrone |
|---|---|
| Dose Level | (mg/m²) |
| 1 | 9 |
| 2 | 12 |
| 3 | 15 |
| 4 | 20 |
| 5 | 25 |
| 6 | 30 |

Three patients are studied at each dose level. Drug administration is repeated every three weeks in the absence of progressive disease or unacceptable toxicity.

Adverse events are graded according to NCI/CTC criteria. Dose-Limiting Toxicity (DLT) is defined as occurrence within the first course of therapy (i.e. 21 days) of unacceptable toxicity, defined as a grade 3 or 4 nonhematologic toxicity including hypersensitivity reactions, other than nausea/vomiting or alopecia or a grade 4 hematologic toxicity other than neutropenia, or a grade 4 neutropenia which persists for more than 3 days or febrile neutropenia defined as grade 3 or 4 neutropenia with a temperature of greater than 38.5° C., or grade 4 vomiting or grade 4 elevation of hepatic transaminases (AST or ALT), or grade 2 (or higher) decline of LVEF following a MUGA scan.

The Maximum Tolerated Dose (MTD) is defined as the highest dose level that causes DLT in no more than one of six patients treated at that level. If none of the initial three patients treated at a given dose level develops dose-limiting toxicity (DLT), dose escalation will continue. If one of the initial three patients treated develops DLT, then three additional patients will be entered on the same dose level. If none of the three additional patients develops DLT, dose escalation will continue. If one or more of the additional three patients treated at a dose level develops DLT, dose escalation will cease. If two or three of the initial three patients treated at a dose level develop DLT, dose escalation will cease. Six patients will be treated at a possible MTD to ensure that criteria are met before declaring that dose level the MTD.

A subsequent course of treatment may be administered 21 or more days after prior liposomal mitoxantrone dose, and when absolute neutrophil count (ANC) is 1,500 m/m³ or more and the platelet count is 100,000/mm³, and recovery from any other treatment-related toxicity (except alopecia) is to baseline grade or less than grade 1, whichever is less restrictive.

Treatment is delayed for one week for resolution of toxicities. If toxicities are not resolved after a one-week delay, treatment will be delayed for one additional week, with the same dose reductions as would have occurred after the one-week delay. If treatment must be held for more than two weeks, then the patient will be removed from the study.

What is claimed is:

1. A method of treating a mammalian disease comprising: administering to a mammal a pharmaceutical composition comprising a therapeutically effective amount of mitoxantrone in a liposomal formulation comprising cardiolipin, and a pharmaceutically acceptable excipient.

2. The method of claim 1, wherein the mammal is a human.

3. The method of claim 1, wherein the liposomal formulation further comprises a phospholipid.

4. The method of claim 1, wherein the liposomal formulation further comprises tocopherol.

5. The method of claim 1, wherein the liposomal, formulation further comprises phosphatidylcholine, cholesterol, and a tocopherol.

6. The method of claim 1, wherein the cardiolipin is selected from the group consisting of natural cardiolipin and synthetic cardiolipin.

7. The method of claim 1, wherein the liposome bears a negative charge.

8. The method of claim 1, wherein the liposome bears a positive charge.

9. The method of claim 1, wherein at least 90% of the mitoxantrone is bound to liposomes.

10. The method of claim 1, wherein the mitoxantrone concentration is in the range of about 0.5 to about 2 mg/ml.

11. A therapeutic mitoxantrone composition comprising a liposome comprising mitoxantrone and a lipid component that contains cardiolipin.

12. The composition of claim 11, wherein the molar ratio of the mitoxantrone to lipid component is in the range of from about 1:10 to about 1:20.

13. The composition of claim 11 wherein the liposome entrapped mitoxantrone comprises vesicles having a size of about 5 μm or less.

14. The composition of claim 11 wherein said liposome entrapped mitoxantrone comprises vesicles having a size of about 1 μm or less.

15. The composition of claim 11 wherein said liposome entrapped mitoxantrone comprises vesicles having a size of about 0.5 μm or less.

16. The composition of claim 11 wherein said liposome entrapped mitoxantrone comprises vesicles having a size of about 0.1 μm or less.

17. The composition of claim 11, wherein the lipid component further comprises a compound selected from the group consisting of phosphatidyl choline, cholesterol, α-tocopherol, dipalmitoyl phosphatidyl choline and phosphatidyl serine.

18. The composition of claim 11, wherein said cardiolipin is selected from the group consisting of natural cardiolipin and synthetic cardiolipin.

19. The composition of claim 11, wherein said liposome bears a negative charge.

20. The composition of claim 11, wherein said liposome bears a positive charge.

21. The composition of claim 11, wherein said liposome is neutral.

22. The composition of claim 11, wherein said liposome is a mixture of multilamellar vesicles and unilamellar vesicles.

23. A therapeutic mitoxantrone composition comprising a lipid component and mitoxantrone wherein the molar ratio of the mitoxantrone to lipid component is in the range of from about 1:10 to about 1:20.

24. The therapeutic mitoxantrone composition of claim 23 wherein the lipid component comprises a phospholipid.

25. The therapeutic mitoxantrone composition of claim 23 wherein the lipid component comprises phosphatidylcholine.

26. The therapeutic mitoxantrone composition of claim 23 wherein the lipid component comprises egg phosphatidylcholine.

27. The therapeutic mitoxantrone composition of claim 23 wherein the lipid component comprises cholesterol.

28. The therapeutic mitoxantrone composition of claim 23 wherein the lipid component further comprises a phospholipid, cholesterol, cardiolipin, and tocopherol.

29. The therapeutic mitoxantrone composition of claim 23 wherein the mitoxantrone concentration is in the range of about 0.5 to about 2 mg/ml.

30. A method for preparing a pharmaceutical dosage form of mitoxantrone comprising the steps of obtaining a vessel containing a quantity of preformed liposomes that comprise a component that binds mitoxantrone, obtaining a vessel comprising a quantity of mitoxantrone in a pharmaceutically acceptable excipient, mixing a portion of the mitoxantrone in the pharmaceutically acceptable excipient with the liposomes, and allowing the mitoxantrone to bind to the liposomes to obtain a pharmaceutical dosage form of mitoxantrone.

31. The method of claim 30 wherein the preformed liposomes are lyophilized.
